(19) United States
(12) Patent Application Publication
(10) Pub. No.: US 2021/0308305 A1
(43) Pub. Date: Oct. 7, 2021

(54) UV SANITISATION DEVICE

(71) Applicants: Emmett Hedigan, Ashford (IE); Bobby CAPRANI, Ashford (IE)

(72) Inventors: Bobby Caprani, Ashford (IE); Jack Gregan, Ashford (IE)

(21) Appl. No.: 17/286,839

(22) PCT Filed: Oct. 21, 2019
(86) PCT No.: PCT/EP2019/078611

§ 371 Date: Apr. 20, 2021

(30) Foreign Application Priority Data
Oct. 23, 2018 (GB) 1817253.6

(51) Int. Cl. A61L 2/10; A61L 2/24

(52) U.S. Cl. CPC A61L 2/10; A61L 2/24; A61L 2202/11; A61L 2202/23; A61L 2202/122; A61L 2202/14; A61L 2202/121

(57) ABSTRACT

The present disclosure is directed towards devices and methods for sanitising a receptacle or asap. In particular, this document describes a sanitisation device comprising a body having an opening configured to receive at least part of a receptacle or tap in use. The body contains one or more UV light sources. The body also comprises a guide, wherein the guide is shaped to position a portion of the surface of the receptacle or tap when the receptacle or tap is inserted into the opening of the body so that the portion is correctly located within the body for irradiation with UV light emitted by the one or more UV light sources, wherein the portion of the surface of the receptacle or tap that the guide is configured to position comprises an opening portion of the receptacle or tap.

### BACKGROUND TO THE INVENTION

The present invention relates to sanitization devices, in particular to devices that use ultraviolet (UV) light for sanitization.

A sanitization device is a device that functions to improve hygienic conditions. The term ‘sanitization device’ is intended to include devices that improve hygienic conditions including but not limited to those that preform disinfection and/or sterilisation. Similarly, things that disinfect and sterilise are specific examples of things that sanitise.

Ultraviolet (UV) light is electromagnetic radiation with a wavelength range of 10 nm to 400 nm. UV light is known to diminish the viability of microorganisms. The term ‘microorganisms’ as used in this document is intended to mean harmful microorganisms such as, for example, germs, bacteria, viruses, and other pathogens.

UV light having a wavelength in the range of 180-280 nm and preferably between 250 and 270 nm has been found particularly useful for sanitisation and has applications including the destruction microorganisms in air, liquids and on surfaces. UV light works as a sanitiser by deactivating the DNA and/or RNA within microorganisms. Specifically, UV light causes damage to the nucleic acid of microorganisms by forming covalent bonds between certain adjacent bases in DNA and /or RNA. Therefore, as the organisms tries to replicate it dies. As a result, as well as destroying microorganisms, UV light inhibits microorganisms' ability to multiply and cause disease.

UV light, and in particular UV light having a wavelength in the range of 180-280 nm, has been used to sterilise equipment and surfaces for a number of applications including biological laboratories and medical centres. A number of everyday products also use UV light to sterilise equipment such as toothbrushes, razors and baby bottles. However, such sterilisation devices typically achieve sterilisation over a relatively prolonged period of time. For example, a toothbrush steriliser may take 7 minutes. Battery-powered sterilisation devices are limited in the intensity of UV light that can be delivered, typically relying on low voltage LED bulbs. There may also be a risk of an operator coming into contact with UV light which leaks out e.g. from a toothbrush holder. The intensity of such UV light should not be high enough to cause damage to users.

Wand-like UV sterilisation devices consist of a long UV lamp, which again produces UV light, attached to a long handle for ease of use. The overall length of the device can vary from small pocket-sized devices having a length of up to 530 mm. These devices are used to sterilise surfaces including countertops, bathroom surfaces, toys, pillows, closets, curtains, desks, telephones, computer keyboards, etc. The wand is held close to the surface and is slowly waved across it. The device is run off rechargeable lithium batteries and can typically sterilise a surface within around 20 seconds. However, the germicidal effect is dependent on the spacing between the wand and the surface being treated and is therefore highly variable depending on how close the wand is held to a surface and how long it is held there. Care must be taken to ensure that the operator's fingers or other body parts are not exposed to the UV light when the wand is being used.

All these previous proposals for using UV light for sterilisation fall into two distinct categories. In the first category the device to be sterilised is placed into a cabinet or holder where the UV light is then applied. In these cases, the user is no longer in contact with the item to be sterilised during the sanitisation operation. In most cases this operation occurs at a slow pace, e.g. over minutes, as there is no urgency for immediate use of the item being sterilised. In the second category the user holds a UV light source and irradiates the item to be sterilised. In this scenario the item to be sterilised usually represents a large area and is sterilised at a distance of many inches resulting in a relatively slow sanitisation e.g. over tens of seconds.

If a user is exposed to immediate or prolonged exposure to UV light, this exposure can result in sunburns, premature skin aging, and even skin cancer. A painful eye injury, called photokeratitis, can also result from an overexposure to UV light. Occupational UV light exposure limits are specified by the EU Optical Radiation Directive 2006/25/EC. This states that the maximum permissible effective radiant exposure value (N_{eff max}) is 30 J/m² within an 8 hour continuous period. This equates to a maximum dosage of 3000 μWs/cm² over an 8 hour period. Hence an operator must not be exposed to high doses of UV light >3000 μWs/cm² in daily life. This can limit the design of UV sterilisation devices that are manually operated in terms of limiting the exposure of an operator to high intensity light. These regulations were brought into law in the UK in April 2010.

For sterilisation purposes, the dosage of UV light received at a surface is a function of the intensity of the UV light received at the surface and the amount of time the microorganisms are exposed to that light (referred to as Residence Time):

$$DOSE = \frac{time ( sec ) \times output ( watts )}{area ( {cm}^{1} )}$$

Some examples of the UV dosage received at a surface to kill some common surface bacteria are shown in the table below:

### Energy Dosage of UV Radiation Needed for Kill Factor (in μWs/cm²)

| | 90% (1 log | 99% (2 log |
|---|---|---|
| Organism | reduction) | reduction) |
| Bacillus anthracis (Anthrax) | 4,520 | 8,700 |
| Bacillus magaterium sp. | 1,300 | 2,500 |
| Bacillus paratyphusus | 3,200 | 6,100 |
| Bacillus subtilis | 5,800 | 11,000 |
| Corynebacterium diphtheriae | 3,370 | 6,510 |
| Ebertelia typhosa | 2,140 | 4,100 |
| Eschrichia coli (E-coli) | 3,000 | 6,600 |

The sterilisation devices that have been proposed for domestic use to periodically clean and sanitise items such as baby bottles, toothbrushes and razors may be able to achieve a high enough dosage to kill E-coli and other bacteria as long as the object being treated is exposed to UV light for a sufficiently long period of time, for example several minutes.

One environment in which sanitisation is desirable is where food and drink receptacles are provided at a point of service e.g. in a bar or restaurant. However, it is desirable for such a sanitisation device to effectively kill microorganisms in a short space of time e.g. a few seconds, for example while a customer is being served or waiting to be served.

Therefore, the present disclosure is directed towards improving sanitisation devices for the provision of food and drink.

In such environments it is important that the period required for sanitisation is short enough not to impede the normal working rate of service. If this period is too long then it is unsuitable for a busy service environment and, if the user has to let go of a receptacle during sanitisation, then there is also potential for further delay. Furthermore, it can be desirable for a customer to witness a food or drink receptacle being sanitised and thus see the added value which the service brings them. This requires that sanitation can occur at the point of service and not at some other hidden location.

The exterior surface of receptacles, including for example receptacles for drinks such as bottles or cans, are typically exposed to contamination by microorganisms when being stored and transported, e.g. being left outside during transport or stored in a cellar. As a result, someone drinking from such a receptacle may ingest the microorganisms on the surface of a receptacle, or the contents of the receptacle may be susceptible to contamination from the microorganisms on the surface of a receptacle upon opening the receptacle. It is therefore desirable to sanitise the surface of such receptacles before they are handed to a customer, especially when the customer may drink or eat directly from the receptacle.

Often this is achieved in a less than ideal way by the bar tender or other serving staff wiping the receptacle with a tissue or damp cloth, e.g. around the top of a can or the neck of a bottle, before handing it to the customer. Such wiping delivers a visually clean drink receptacle but does not remove the microorganisms on the outer surface of the receptacle which may have accumulated at any point on its journey to the customer after having been filled and sealed e.g. at a brewery. Indeed, a tissue or damp cloth can easily become contaminated by microorganisms. As a result, wiping a receptacle in this way may simply be further source of microorganism contamination.

Drinks that are dispensed at a point of service, for example from a tap or faucet, are also prone to contamination because the tap is typically in regular use and often cleaned infrequently. For beverages such as beer, the tap may even be immersed into the liquid it is dispensing as it is being dispensed. Microorganisms can readily build up in, and on, the tap. At present there is no convenient way to sanitise a dispensing tap without disrupting its use. Furthermore, beverages such as beer may be pumped through to the dispensing tap from a keg and any couplers used between the keg and the supply line may also pose a risk of contamination. Again, there is no quick way to sanitise a keg coupler or tap system when a beer line is swapped between kegs.

Therefore, there is a need to solve these and other sanitisation problems.

### SUMMARY OF THE INVENTION

The present disclosure is directed towards devices and methods for sanitising a receptacle or a tap, the features of which are set out in the appended claims. According to a first aspect of the present invention there is provided a sanitisation device comprising a body having an opening configured to receive at least part of a receptacle or tap in use. The body contains one or more UV light sources. The body also comprises a guide, wherein the guide is shaped to position a portion of the surface of the receptacle or tap when the receptacle or tap is inserted into the opening of the body so that the portion is correctly located within the body for irradiation with UV light emitted by the one or more UV light sources, wherein the portion of the surface of the receptacle or tap that the guide is configured to position comprises an opening portion of the receptacle or tap.

As used in this document, the word ‘receptacle’ refers to a hollow object that can be opened which used to contain something. As such, a receptacle for providing food or drink is any object having a hollow space which can be used to contain food or drink. For example, a drink receptacle cold be any one of: a bottle (such a glass bottle (e.g. a beer bottle), a reusable plastic bottle, a disposable plastic bottle, a baby bottle, etc.); a flask; a carton; a can; etc. A food receptacle could be a can, a carton, a jar, a flask, a bottle (e.g. for yogurt or soup), etc.

As used in this document, the word ‘tap’ refers to a device which controls the flow of a fluid from a pipe or container. For example, a tap can control fluid flow by having an open setting which allows fluid to flow from the device and a closed setting which prevents fluid flowing from the device. A tap can be, for example: a device such as keg coupler (a keg coupler can also be referred to as a keg tap head), a valve, a spout, a stopcock, a cock, a spile, a faucet, a spigot, etc.

The opening portion of the receptacle or tap is a portion of the receptacle which comprises an opening through which food or drink can be accessed upon opening the receptacle or tap, e.g. the portion of a receptacle configured to accept a cap, lid or other closure, a tap head, etc.

Preferably the body is a sleeve and more preferably an elongate sleeve. It will be understood that such a device is in some embodiments the device is configured for use with one or more types of receptacle. In particular the device may be configured to receive glass bottles. For some of these embodiments, the device is designed for a bar tender or other user to insert the neck of a bottle just before serving it to a customer, the sleeve-like configuration enables rapid insertion of the bottle for irradiation with UV light from the UV light source(s) while helping to protect the user from exposure to UV light. Accordingly, sanitisation can take place while the user is still holding the receptacle.

The guide may be dimensioned so as to have internal dimensions suitable for guiding or closely matching those the opening portion of one or more different types of receptacles.

For example, of a range of bottles sizes, as the upper neck of a drinks bottle, especially a glass bottle, typically has a standard diameter corresponding to the metal “crown cork” cap. Moreover it will be appreciated that the distance between the exterior surface of the opening portion of such a glass bottle the UV light source(s) may therefore be kept within a narrow range regardless of the type of bottle.

Preferably the guide is arranged to guide the opening portion of a receptacle or a tap so that the exterior surface of the opening portion is located within a range of 25 mm or less from the UV light source(s). More preferably the opening portion is guided so that its exterior surface is located within a range of 20 mm, 15 mm, 10 mm, 5 mm or less. The close distance between the surface to be treated and the UV light source(s) ensures that there is achieved a high intensity of UV light reaches the surface of the opening portion. As only 25-50 mm of the receptacle or tap need to be sanitised (e.g. for a drink receptacle, the opening portion and ideally the part of the receptacle in contact with the mouth of a user drinking from the recptacle or the part of a tap immersed in a beverage e.g. beer), the important parameter is the intensity of UV light reaching the surface of the opening portion of the receptacle or tap. The intensity of UV light reaching the opening portion of the receptacle or tap from the UV light source(s) is increased by reducing the distance between the UV light source(s) and the opening portion of the receptacle or tap. For example, when using standard UV light bulbs that deliver an output of 7 μW/cm² (at a distance of 1 m), a dosage sufficient to kill E-coli and other pathogens can be delivered in around 5 seconds or less by reducing the distance to the surface e.g. to 20 mm so that the intensity is increased by a factor of 180 to more than 1000 μW/cm². The device therefore allows a bar tender or other user to quickly sanitise a receptacle or tap at the point of service e.g. in front of a customer.

As the one or more UV light source(s) are preferably arranged close to the surface of the opening portion of the receptacle or tap to minimise sanitisation time, the guiding means inside the sleeve helps guide the surface to be sanitised into a position close to the UV light source(s) while protecting e.g. the UV light source(s) to ensure that the receptacle or tap does not strike and damage or break the one or more UV light sources. Such guiding means can also help to ensure that the surface is positioned at a close distance (e.g. less than 25 mm) regardless of the overall dimensions of receptacle or tap.

The UV light source(s) can be any suitable source of UV light. For example, a UV light source may be a fluorescent lamp, a mercury-vapor lamp, a light-emitting diode (LED), a lasers (suitably rastered), or an incandescent lamp. The UV light source(s) may optionally be provided with one or more light filters, either on the UV light source or in a separate glass filter, which blocks at least some visible light and allows through UV light.

The device may be battery-operated e.g. the UV light source(s) comprising lower voltage UV LEDs, but the UV light delivered will be of relatively low intensity. However, this can enable the device to be cordless and portable for ease of handheld use. In at least some embodiments the device may be a portable device comprising a battery, preferably a rechargeable battery. The device may include a mains operated charging dock to recharge the battery when the portable sanitisation device is connected thereto. Any suitable kind of electrical connection may be provided, such as a plug connector or inductive coupling.

In at least some embodiments it is preferred that the device is mains operated, so that higher voltage UV light source(s) may be used. UV light source(s), such as e.g. UV bulbs, are typically rated by Watts of UV power outputted (sum over all directions) and/or Watts per cm² (for a surface 1 m away from the bulb). The UV source(s) in the device preferably has a rating of at least 5 μW/cm², preferably around 7 μW/cm², and further preferably greater than 10 μW/cm² or even 20 μW/cm².

For the device to use UV light source(s) currently available on the market, e.g. rated at around 7 μW/cm², it is preferable to arrange the light source(s) so as to maximise the intensity of UV light delivered to the surface around the opening portion of the receptacle or tap. In other words, it is preferred that the UV light source(s) is/are arranged to provide multi-directional irradiation. In one set of embodiments there is provided an array of multiple UV sources. The device may therefore comprise at least two, three, four, five, six or more UV light source(s) arranged inside the sleeve.

Preferably, UV light bulbs are used as the UV light source(s). Preferably, a circular or one or more annular UV light bulbs may be provided so as to surround the opening portion of the receptacle or tap. However such curved bulbs may involve higher manufacturing costs. Alternatively the UV light bulbs can, for example, be an array of linear bulbs. The multiple UV light bulbs may be arranged circumferentially and/or axially inside the sleeve.

In another set of embodiments, alternatively or in addition, there is provided reflection means for the UV light source(s). The reflection means may comprise a parabolic mirror or reflective surface or array of mirrors/reflective surfaces e.g. with a UV light source and opening portion of the receptacle or tap positioned at or close to the focal points to maximise surface intensity. By arranging multiple UV light sources and/or reflection means to irradiate the surface of a receptacle or tap with an increased intensity of UV light, a radiation dose sufficient to kill harmful bacteria can be delivered in a few seconds. Fast sanitisation is therefore assured.

The device is preferably designed to deliver an intensity of UV light at the surface of the opening portion of the receptacle or tap that is at least 500 μW/cm², preferably at least 1000 μW/cm² and further preferably in the range of 1000-10,000 μW/cm², for example 1000-5000 μW/cm². With such intensities the dose after 1-10 seconds can be sufficient to kill bacteria such as E-coli and those causing Weil's disease.

The illustrative examples of sanitisation devices discussed above use a body having the form of an elongate sleeve which is preferable to accommodate a bottle neck or beer tap. However, the body may be any suitable shape. Furthermore, the guide can be shaped to work with other food and drink receptacles and other taps as described below with reference to the figures.

The device may also be used to sanitise portions of other objects that fit inside the body, for example at least part of a keg coupler or tap head which engages with a keg or beer line. A benefit of body having the shape of an elongate sleeve is that the UV light source(s) can be positioned away from the open end of the sleeve to reduce the risk of radiation leakage. However, it will be appreciated that a non-elongate sleeve or other opening may also be shaped so as to accept an object to be sanitised while remaining safe for handheld use. Such a sanitisation device may have an opening and guide more suitably shaped to accept a particular object, such as a keg coupler or tap head.

Thus according to a further aspect of the present invention there is provided a sanitisation device comprising an opening and a guide shaped to accept a keg coupler. The keg coupler can be manually be inserted into the opening. The opening leads to a treatment zone containing one or more sources of UV light arranged to irradiate the surfaces of the keg coupler.

A keg coupler is the device which is connected to a valve to a kegs to connect the keg to a dispensing tap e.g. via a beer line in a pub. The keg coupler can be is a considerable source of contamination, and therefore may optionally need to be cleaned before use. By using a device to add sanitisation to the process of connecting beer lines, there can be ensured a high level of cleanliness, thus minimising contamination and lengthening the period between maintenance cycles.

Preferably, the sanitisation device is configured to be a wall mounted device. In this embodiment, a keg coupler can be removed from an empty keg and placed in the sanitisation device. As a result, the keg coupler will undergo sanitisation while the user is replacing the empty keg with a replacement keg. This is advantageous because the keg coupler will be sanitised and ready to be fitted to the replacement keg when the replacement keg is in position, which means that the sanitisation of the keg coupler comes with no time cost to the user who is replacing a keg.

As will be recognised by the person skilled in the art, in this embodiment it is preferable for the user not to have to hold the keg head in position during sanitisation. Therefore, the device may be provided with a holding means to hold the opening portion of the keg head in position during sanitisation. The holding means can be provided by shaping the guide so that it holds the keg head in the correct position under gravity. Alternatively, the holding means may be provided by providing the guide with a spring lock. The spring lock is configured such that pushing the keg coupler into to the correct position engages the spring lock which then holds the keg coupler in the correct position. The keg coupler is released from the sanitisation device by pushing the keg coupler again, which disengages the push lock. As a further low-cost alternative, the holding means may be provided by a strap which can be fixed to the sanitisation device to hold the keg coupler in place.

Alternatively, the sanitisation device can be a handheld device. The sanitisation device may also optionally be cordless and portable. These features may be advantageous for home use or for hobby craft brewers, where equipment needs to be stored away after use.

The opening and guide may have any shape suitable for one or more standard tap head designs, which may vary to accommodate different types of keg valves, such as D-system, S-system, U-system, A-system and G-system. The opening may be shaped to accept a keg tap head including a U-shaped keg valve (e.g. for Guinness) or a G-system coupler (e.g. for Boddingtons), or a European Sankey keg tap with an S-shaped keg valve (e.g. for Heineken) or a US Sankey keg tap with a D-shaped valve (e.g. for US Budweiser). Of course, keg tap heads can come in a variety of shapes and sizes, both to fit the different types of valves and depending on style.

While it is beneficial to deliver a high intensity of UV light to reduce the sanitisation time, the Applicant has recognised that safety features may be required to protect users of the device, especially when the sleeve or opening is designed for manual insertion of a receptacle to be treated or manual manipulation to slide over a tap or keg coupler. In one set of embodiments the UV light source(s) are arranged so as to direct radiation away from the open lower or upper end of the sleeve. In other words, the opening portion of a receptacle inserted into the sleeve is irradiated from below so that a hand gripping the bottle is not exposed to the UV light.

Similarly, where the device is held to insert a tap through the open upper end of the sleeve the UV light does not escape out of the sleeve. However, there may still exist a risk of UV light being reflected down or up towards the open end of the sleeve. The same issues may exist for any other shaped opening. One way to protect a user from such stray UV light could be to provide gripping means for the receptacle or tap so that a user does not have to hold the receptacle or tap during treatment but can move their hands away. However, this is likely to add to the overall treatment time as well as the complexity of the device. It is therefore preferable that the device comprises a shielding means arranged to prevent UV light from reaching the open lower or upper end of the sleeve or the opening. The shielding means may advantageously allow a user to manually insert a receptacle or tap into the sleeve without coming into contact with UV light even if the UV light source(s) are operational at the time of use. The shielding means be separate from the guiding means mentioned above, or a single structure may provide both guiding and shielding functions. The guiding and/or shielding means may be formed from, or comprise, a deformable or resilient material so that it better conforms to various different shapes of receptacles or taps being inserted. The shielding means may be combined with one or more other safety features, as will be described below.

It will be appreciated that a sanitisation device which allows a user to manually insert and remove a receptacle or tap after only a few seconds of treatment, combining a high UV light dosage with operational safety, is unique as compared to existing devices. The only sanitisation apparatus that can rapidly kill dangerous bacteria such as E-coli in a relatively short space of time are similar to an autoclave in design, i.e. a closed treatment chamber. While such apparatus provides a high degree of safety, rapid treatment is not a possibility as any item to be treated must be placed in the chamber, the chamber sealed closed, and then a user waits for treatment to be completed before opening the chamber and removing the sanitised item. Therefore, there remains a need for a device that can provide rapid sanitisation and ease of use together with safety for users.

This feature is considered novel in its own right and thus when viewed from a further aspect of the present invention there is provided a sanitisation device comprising a treatment zone provided with one or more sources of UV light arranged to irradiate the exterior surface of an item positioned therein, wherein the treatment zone is partly open to allow an item to be treated to be manually inserted and held in position in the treatment zone, the device further comprising shielding means to prevent UV light from leaving the treatment zone. Such a device enables rapid manual operation, with a user being able to insert and remove an item for treatment with their hand remaining close to the open treatment zone but protected from exposure to UV light by the shielding means.

Embodiments of this aspect of the invention may be suitable for sanitising a wide range of items, including items for medical use. In a preferred set of embodiments the treatment zone is designed to be partly open for the insertion of a food or drinks receptacle. The device is ideally suited for manual use at the point of service in a shop, bar, restaurant, cafe, etc. In one set of embodiments the treatment zone comprises an elongate sleeve open at a lower end for opening portion of a receptacle or tap to be manually inserted from below.

The features described hereinabove in relation to the first and second aspects of the invention are equally applicable to these further aspects of the invention. In particular, the UV light source(s) are preferably arranged to irradiate the exterior surface of an item positioned in the treatment zone at a distance of 20 mm, 15 mm, 10 mm, 5 mm or less. The treatment zone may comprise multiple UV light sources and/or reflecting means as described above. Preferably the device is designed to deliver an intensity of UV light at the surface of an item positioned in the treatment zone that is at least 500 μW/cm², preferably at least 1000 μW/cm² and further preferably in the range of 1000-10,000 μW/cm². With such close distances and/or high intensities the dose of UV light delivered in a few seconds can be enough to kill many dangerous microorganisms.

While the shielding means is preferably designed such that a user may safely hold a receptacle or tap in position in the treatment zone without being exposed to UV light, this may rely on a user holding the receptacle or tap properly outside the treatment zone. If a user were to insert part of their hand e.g. fingers into the treatment zone along with, or instead of, an item then they could potentially be exposed to dangerous levels of UV light. It is therefore preferable for the device to comprise one or more further safety features, which may be provided in combination with (or instead of) the shielding means.

In one set of embodiments the device comprises a means for detecting the presence of an item in position the treatment zone. The means for detecting may comprise one or more sensors arranged in the treatment zone away from the open part so as to detect when an item has been properly inserted but not to detect a stray finger or the like. The means for detecting may be connected to a controller or power circuit for the UV source(s) such that the UV source(s) are not operated until the means for detecting provides an indication that an item to be treated is positioned in the treatment zone. The means for detecting may be a switch or a sensor. For example, a microswitch or pressure sensor may indicate when item is in position. Where the treatment zone is located in the interior of the sleeve, the microswitch or pressure sensor may be located distal from the open end to detect when a the opening portion of a receptacle or tap has been fully inserted into the sleeve. The use of such means for detecting may help to ensure that the UV light source(s) are only operated when the opening portion of the receptacle or tap is positioned at a certain distance from the UV light source(s), for example with a surface to be treated within a range of 25 mm or less. The means for detecting may help to minimise unnecessary power consumption by the UV light source(s) so that they are only operated as and when required. Furthermore, the means for detecting may provide for automatic operation of the UV light source(s) without a user needing to turn the device on for treatment to commence, helping to reduce the time taken for a treatment cycle.

At least one means for sensing a hazard may optionally be arranged to detect the presence of an item that is not suitable for treatment, for example a sensor such as an infrared sensor may be used to detect when a warm body part, such as for example a user's finger or hand, is in the treatment zone. Such a means for sensing a hazard may be connected to a controller or power circuit for the UV light source(s) such that the UV light source(s) cannot be operated while a human presence is detected in the treatment zone. Of course more than one means for sensing a hazard may be provided for multiple levels of safety.

Alternatively, the guide can be configured such that it is not possible for a human hand to physically access the treatment zone. For example, the guide can have opening and a length which mean a human hand cannot fit far enough within the guide to reach the treatment zone.

There will now be described some further features that may be applied to embodiments according to any aspect of the invention. The sanitisation device is preferably capable of being counter top-mounted, for example for ease of use by a server in a bar, restaurant, etc. and for visual sanitisation verification by the customer. Any combination of one or more of visible light (i.e. electromagnetic radiation having a wavelength in the range of 400-700 nano-meters) from a visible light source, fluorescent materials that absorb UV light and emit visible light, and/or non-UV transparent materials can be used to deliver a visual indicator of the sanitisation event. A front mounted display can alternatively or additionally be added to advertise to or inform the customer.

In some embodiments it may be useful for the device to be easy to clean and maintain in its working environment. All internal materials (e.g. the sleeve) need to be easily washable as froth and spills from receptacles may occur. Optionally the sleeve or other structure forming the treatment zone can be detached and cleaned with a safety feature implemented to ensure the UV light source(s) cannot operate without the sleeve being safely and correctly inserted.

Alternatively, the device can be formed as a single unit thereby reducing the cost of the unit.

According to a further set of embodiments of the invention the device may be in incorporated into a dispenser machine for dispensing receptacles, e.g. a drink receptacle dispenser such as a bottle or can dispenser machine, preferably arranged so as to provide rapid sanitisation of the receptacle on delivery. Such embodiments also take advantage of the sanitisation event being fast with close positioning of the UV light source(s) to the surface to be sanitised. Thus, sanitisation may occur before the user takes the receptacle from the serving position. One implementation may involve a closure or blocking of the serving position from the user until after sanitisation, especially where a risk of exposure to UV light exists. A non-UV-transparent plastic sleeve that blocks UV light while allowing visible light to pass or other suitable cover for the treatment zone could be used, allowing the user to see the sanitisation event safely.

According to a further set of embodiments of the invention the device may be mounted to a food or drinks cabinet where the user manually uses the sanitisation device after selection of a product. Such an implementation could be similar to the prior implementations where the user never lets go of the receptacle, but inserts one end of the product into the sanitisation device for rapid sanitisation, or alternatively the user may let go of the product during the rapid sanitisation with the sanitisation location being closed from the user during the rapid sanitisation event.

According to a yet further aspect of the present invention there is provided a method of sanitising a receptacle or tap, comprising: manually inserting the receptacle or tap in a partially open enclosure and allowing UV light to irradiate the exterior surface of the receptacle or tap while it is positioned in the partially open enclosure. According to another aspect of the present invention there is provided a method of sanitising a tap such as a drinks dispensing tap or faucet, comprising: manually positioning a partially open enclosure over a tap and allowing UV light to irradiate the exterior surface of the tap while it is positioned in the partially open enclosure. Preferably the receptacle or tap is inserted so as to be located within a close range of one or more UV light source(s), as described above. Further preferably the sanitisation takes place while a user is still holding the receptacle or tap, or holding a sanitisation device over the receptacle or tap, although shielding means may ensure that the user is not exposed to the UV light.

In some embodiments a wavelength around 184.9 nm may be chosen so as to create ozone, which can also kill microorganisms. UV source(s) operating at different wavelengths may be combined in the same device to provide multiple anti-microbial effects.

### BRIEF DESCRIPTION OF THE DRAWINGS

Some exemplary embodiments of the present invention will now be described with reference to the accompanying drawings, in which:

FIG. 1 is a side view of a sanitisation device;

FIGS. 2a and 2b are bar-side views of a sanitisation device;

FIG. 3 is a schematic view of some internal features of a sanitisation device;

FIG. 4 illustrates a first example of a UV light source;

FIG. 5 illustrates a second example of a UV light source;

FIG. 6 illustrates a side cut-away view of sanitisation device having a moveable guide with the moveable guide in a first position;

FIG. 7 illustrates a side cut-away view of sanitisation device having a moveable guide with the moveable guide in a first position engaging with a bottle;

FIG. 8 illustrates a side cut-away view of chamber sanitisation device having a moveable guide with the moveable guide in a second position engaging with a bottle;

FIG. 9 is a front-isometric view of a dual guide sanitisation device;

FIG. 10 is a back-isometric view of a dual guide sanitisation device;

FIG. 11 is a perspective view of a typical keg coupler or tap head;

FIG. 12 is a side cross-sectional view of an alternative sanitisation device;

FIG. 13 shows the relationship between the intensity of UV light and the distance an object is from the source;

FIG. 14 shows the spectral distribution of germicidal effectiveness against the wavelength of UV light; and

FIG. 15 shows the energy levels of UV light required for the destruction of 99.9% (log 3) of various microorganisms at 254 nm wavelength.

### DETAILED DESCRIPTION OF THE DRAWINGS

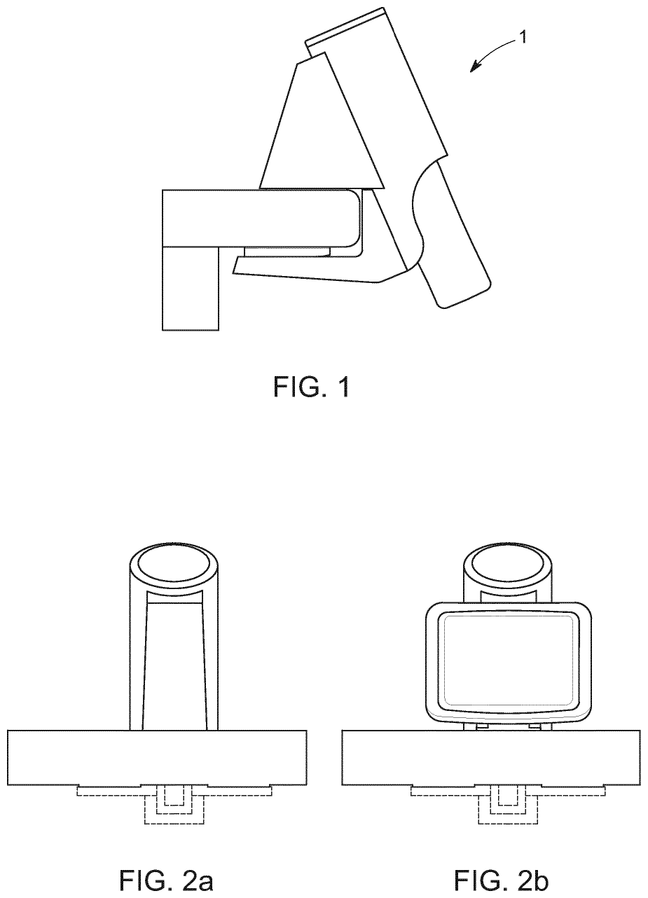

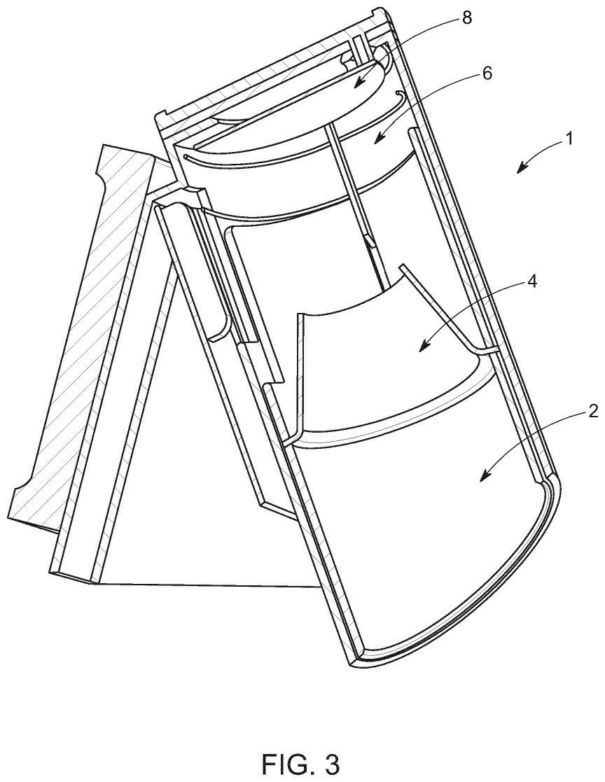

FIGS. 1 and 2 show one configuration of a sanitisation device 1 comprising a clamp. In

FIGS. 1 and 2 the sanitisation device 1 is attached to a bar with the clamp. The clamp is designed so as not to damage the bar and can easily be removed.

From the side view of FIG. 1 it is seen that a receptacle can be inserted into a sleeve 2 of the device 1. In use, for a drink receptacle the opening portion of the receptacle will come into contact with the mouth of a user when liquid is drunk from the receptacle. It is therefore preferable that at least the opening portion (e.g. in the case the receptacle is a beer bottle, the upper neck region of the bottle) is irradiated with UV light.

As is seen from FIGS. 2a and 2b, an optional feature is the use of an advertisement label or digital image at the front of the device 1, facing the customer.

FIG. 3 shows a schematic of the internal features of the device 1. The device 1 provides a sleeve 2 (of which the back half is shown) for receiving a receptacle. The sleeve 2 is provided internally with a guide 4 which allows the receptacle to be brought into the correct location within the device. Those skilled in the art will recognise that the sleeve guide 4 can be shaped to guide an opening portion of one or more different types of food or drink receptacles. As a result, when the correct type of receptacle (i.e. a receptacle having an opening portion which the sleeve guide 4 is shaped to guide) is inserted into the sleeve 2, the guide 4 will correctly position the opening portion within the sleeve 2 for sanitisation with UV light. For the purposes of illustration, a beer bottle is used as an illustrative example of a receptacle in the figures. However, those skilled in the art will note that the example of a beer bottle is not limiting. For the sake of completeness, it is noted that the opening portion of a receptacle is a portion of the receptacle comprising an opening.

As a result of using the sleeve guide 4, precise placement of the receptacle by the user is not necessary. Furthermore, the conical shape of the guide 4 provides a shield to the user to minimise UV light that might otherwise travel towards the user's hand. Indeed, a key consideration is to minimise any risk of the UV light shining onto a user's hand when holding the receptacle. The receptacle may itself be made of glass configured to block any UV light shining through it and thus only the space around the receptacle needs to be shielded to minimise egress of UV light.

A space 6 is provided for the placement of one or more UV light sources and/or reflectors, mirrors or the like (not shown). Ideally these are designed and/or arranged to minimise egress of UV light from the opening. A pressure sensor 8 is configured to activate an ON switch in response to pressure from the receptacle. The UV light source(s) generate UV light when the ON switch is activated. This is a safety feature to ensure that sanitisation only occurs when a receptacle such as a bottle, etc. is properly inserted. Other sensors can be located in the sleeve 2 to enhance the safety and functionality. For example, an infrared sensor could detect whether a warm object (e.g. a user's finger) is inserted in the sleeve 2 and the device can be configured to prevent activation of the UV light source(s) upon detection of the warm object. Another possibility is to provide contact sensors on the sleeve 2 to ensure that the receptacle is positioned against the guide 4 in the correct manner. Another optional sensor could be a tilt switch to ensure the device 1 is correctly mounted.

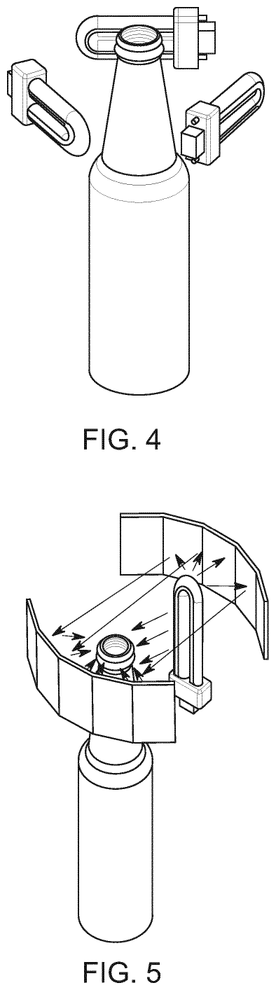

FIGS. 4 and 5 show two different scenarios to ensure that the surface of an opening portion of a receptacle (for example, the cylindrical surface of the upper portion of the neck of a bottle shown in FIG. 4) is sanitised. In FIG. 4 this is achieved by positioning multiple UV light sources (for example UV lamps) around the opening portion of the receptacle to be sanitised. Germicidal UV bulbs may optionally be used. Germicidal UV bulbs come in many different shapes and sizes, with most being in the shape of either a single or double tube.

In FIG. 5 mirrors (or other reflectors) are positioned to reflect the UV light from a single UV light source towards the surface of an opening portion of a receptacle. However, the embodiments shown in FIGS. 4 and 5 can be combined and mirrors (or other reflectors can be used in combination with any suitable number of UV light sources.

Other UV light source configurations are possible. UV light bulbs having different shapes, e.g. one or more circular bulbs can also be used. What is important is that the UV light source(s) are configured to deliver a high enough concentration of UV light to sanitise at least an opening portion of the surface of a receptacle.

The intensity of UV light delivered to of the surface of the opening portion of a receptacle can be increased by reducing the distance between the UV light source and the opening portion. As a result, the device is preferably designed so that the guide positions the surface of the opening portion of the receptacle at a distance of 25 mm or less from the UV light source(s).

Increasing the amount of UV light delivered from the UV light source(s) to the surface of the receptacle to be sanitised reduces the time required for sanitisation. As a result of the short distance between the UV light source(s) and the surface of the receptacle to be sanitised, guide is also important to prevent the receptacle coming into contact or striking the UV light source(s).

With further reference to FIG. 5, the mirror can, for example, be a parabolic mirror arrangement comprising one or more parabolic mirrors. In the example shown in FIG. 5, a UV light source is located in the focal point of a first parabolic mirror and a second parabolic mirror is positioned facing the first parabolic mirror so that UV light reflected by the first parabolic mirror is directed onto the second parabolic mirror. The guide is configured to position the surface of the opening portion of the receptacle to be sanitised in the focal point of the second parabolic mirror. While using a typical 8.5 cm long UV bulb as a UV light source, 1.2 W of UV light may be delivered on to a surface 50 mm away.

This dosage is increased by using reflectors as shown in FIG. 5. For a typical beer bottle neck, the surface area to be irradiated with UV light is around 8 cm². The reflector design of FIG. 5 is able to capture over 33% of the light from the source:
Total UV Energy in bulb=1.2 W
Energy over 1 cm of bulb height=1.2 W/8.5 cm=141 mW
Energy being reflected onto product=141 mW*33%=47 mW
Energy per cm² of product surface=47 mW/8 cm²=5880 μW/cm².

A good reflector design in combination with positioning of the surface to be treated can therefore result in higher intensities of UV light enabling faster sanitisation to be achieved e.g. (sanitisation can be achieved in only one or two seconds).

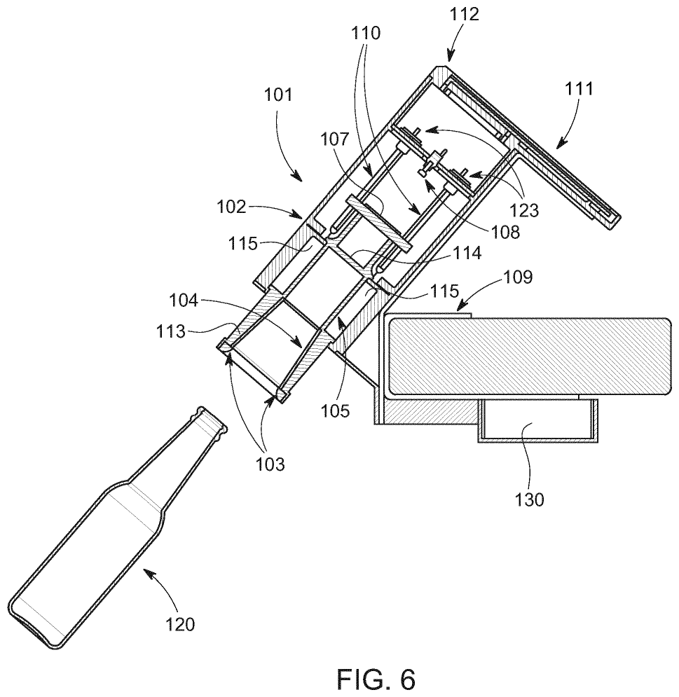

FIG. 6 shows another embodiment of a device 101 for sanitising at least the surface of an opening portion of a receptacle. The device 101 comprises a sleeve 102. The sleeve 102 comprises a mounting plate 123. A plurality of UV light sources 110 are mounted on a mounting plate 123. The sleeve can be formed or assembled as a single part to keep costs down. Alternatively, the sleeve can be provided with a removable closure, where the UV light source can be accessed by removing the closure.

A switching means 108 is also mounted on the mounting plate. The switching means is moveable between an on position and an off position. Preferably, the switching means 108 is a push switch, wherein the switch is movable at right angles to the mounting plate between the off position and the on position, wherein the on position is located closer to the mounting plate than the off position. When a receptacle is fully inserted into the sleeve 102, the receptacle forces the guide to push the switching means into the on position. While in the on position, the switching means activates a timer. The timer measures the time that the switching means is maintained in the on position. If this time exceeds the amount of time required to sanitise the receptacle, an audio and/or visual alert and/or alarm can alert the user that sanitisation of the receptacle is complete. In one embodiment, the alert can take the form of a progress bar on a display. If the switching means switches to the off position before the timer measures the amount of time required to sanitise the receptacle, this indicates that the receptacle has been withdrawn from the sleeve too early and the device can be configured to provide an audio and/or visual alert and/or alarm to the user to indicate that sanitisation has not been correctly performed.

At present, it takes a certain amount of time to activate the most suitable UV light sources for sanitisation. Therefore, in the embodiment shown in FIGS. 6-8 the UV light is constantly on in use (i.e. the UV light is on even when no receptacle is inserted into the sleeve). This advantageously reduces the treatment time as the treatment time will not include the time required to activate the UV light source(s).

However, as e.g. UV LEDs improve, it is envisaged that the activation time of UV light source(s) will decrease and eventually become negligible. It is therefore possible to optionally configure the switching means to control the UV light source(s) as well as the timer. When the switching means is in the on position, the UV light sources 110 can be activated to generate UV light. When the switching means is in the off position, this means that the receptacle has been removed from the sleeve and the switching means can be configured to deactivate the UV light source.

The UV light sources are preferably germicidal UV light bulbs. The interior of the sleeve 102 can optionally be coated with a reflective material. One or more vent(s) (not shown) are optionally provided to allow any heat generated by the UV light sources to dissipate.

The device is also provided with one or more controls and a display on an external mount 111. Preferable the one or more controls and the display are integrated using a touch screen display. The display can be used to provide an indication that sanitisation of a receptacle has been completed and/or an indication of the remaining time required to achieve the desired amount of sanitisation. The controls can optionally be used to adjust the how the device operates.

The sleeve 102 is provided with a bracket 109 for mounting on a bar or table. The bracket can be tightened onto the bar or table using a screw clamp or the like. The bracket also comprises a space 130 where a power adaptor (not shown) is located. The power adaptor is configured to convert electricity provided by the mains to a voltage suitable for powering the UV light sources. A battery (not shown) can also be provided in space 130 as an auxiliary or alternative power supply.

The sleeve 102 is also provided with a moveable guide 104 for a receptacle. The moveable guide 104 shown in FIGS. 6-8 is shaped to receive a beer bottle. However, those skilled in the art will note that this example is not limiting, and the guide 104 can be shaped to receive different types of receptacles. For example, the guide 104 could also be shaped to receive a can as previously described above.

Where, as shown in FIGS. 6-8, the guide 104 is shaped to receive a bottle, the guide is provided with a neck shade 113 the neck shade can be made of or coated with a material which blocks UV light. Alternatively, the neck shade 113 can be provided by coating a portion of the guide 104 with a paint or material which blocks UV light.

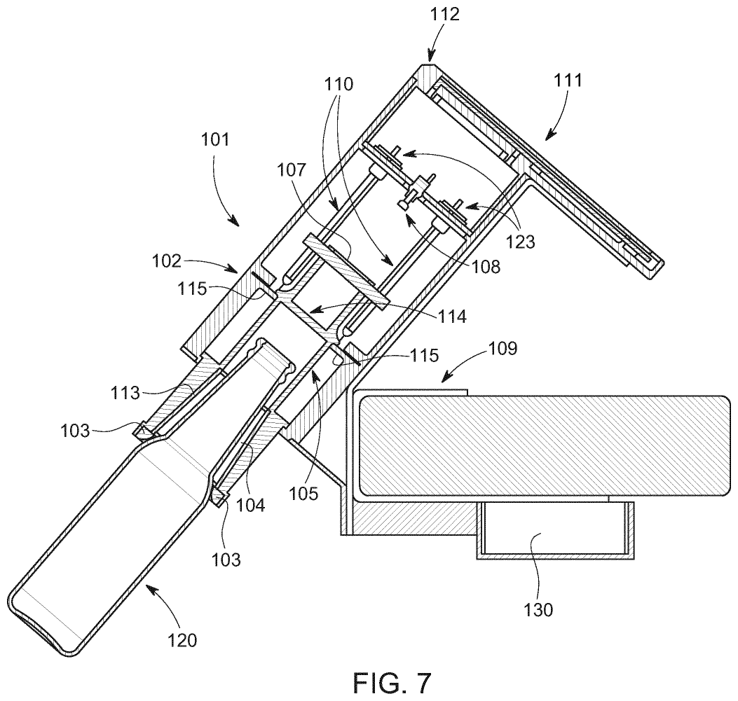

The mouth of the guide is provided with a ring 103 of a resilient material, such as rubber. As shown in FIG. 7, the ring 103 of a resilient material makes contact with the receptacle in use, preferably forming a soft seal with a portion of the surface of the receptacle. The guide 104 has a chamber 105 which is made from a transparent material such as for example quartz glass.

The guide 104 is configured to move between a closed position and an open position as will be described below in more detail. The guide 104 is biased towards a closed position as shown in FIGS. 6 and 7.

The guide 104 can also be provided with cap 114 which is made from a material or coated with a paint or material that blocks UV light. As shown in FIG. 6, when the guide 104 is in the closed position, the cap 114 is pressed against a lip 115 inside the sleeve. This blocks UV light present in the sleeve 101 from exiting the sleeve when the devise is not in use.

As shown in FIG. 7, in the case where the receptacle is a bottle 120, the guide 104 is shaped such that when a soft seal is formed between the ring 103 and the bottle 120, the top of the bottle 120 extends beyond the neck shade 113. For other types of receptacle it is envisaged that a soft seal is formed between a ring in the guide and the opening portion of the receptacle.

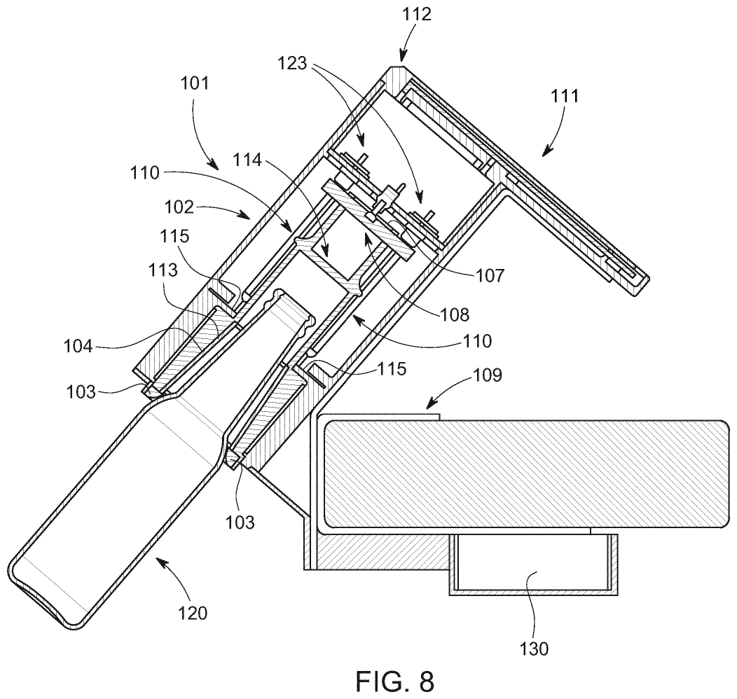

As shown in FIG. 8, pushing the receptacle further into the sleeve causes the moveable guide 104 to move to a closed position. In this closed position, a plate 107 on the guide 104 is pressed against push switch 108 forcing the push switch to the on position, thereby activating the timer. Simultaneously, the opening portion of the receptacle to be sanitised is moved into a position in the sleeve where it is exposed to UV light. UV light is prevented from exiting the sleeve between the receptacle and the guide 104 by the ring 103.

Where, as shown in FIG. 8, the receptacle is a bottle, the top of the bottle 120 is exposed to UV light, while UV light is prevented from exiting the sleeve via the neck of the bottle 120 (if the bottle is made of a transparent or opaque material) by the neck shade 113. Ideally, the UV light sources 110 are positioned within the sleeve such that the angle between the axis running along the length of the bottle and any UV light emitted from the UV light sources 110 entering the top of the bottle is large enough to cause any UV light entering the bottle 120 to be absorbed by the neck shade 113.

The cap 114 can also function to minimise the UV light reaching the bottle 120 that was emitted from the UV light sources 110 at a small enough angle to exit the bottle 120 in the absence of the cap 114.

Furthermore, the UV light sources 110 are also positioned such that, in the case where the receptacle is a bottle 120, the inside of the mouth of the bottle is exposed to UV light. This allows the bottle 120 to inserted into the sleeve and sanitised after opening, reducing the risk of the liquid in the bottle 120 being contaminated by microorganisms after opening.

A further advantage of the device shown in FIGS. 6-8 is that the length and the narrowness of the space within the moveable guide 104 prevent a user's hand from fitting far enough into the guide to reach a position where it is exposed to UV light—even if they force the moveable guide 104 into the closed position.

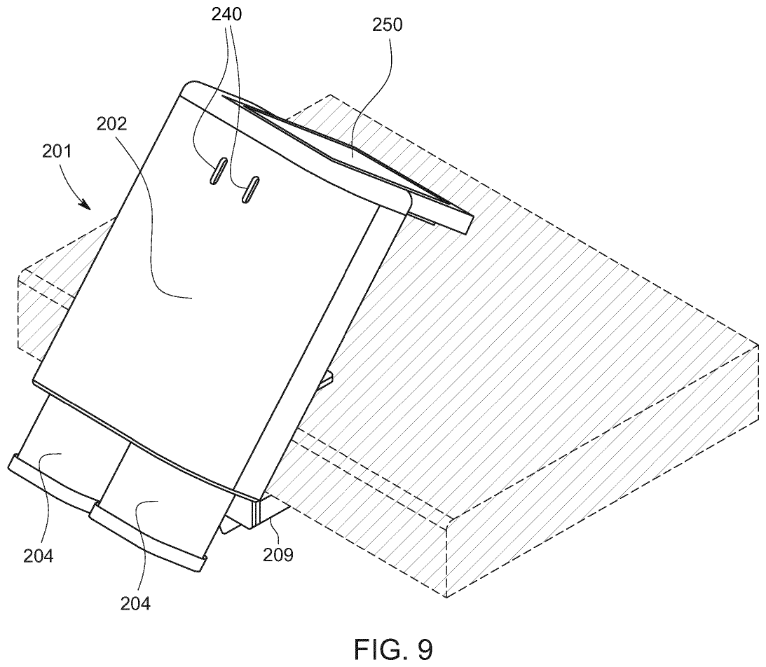

FIG. 9 is a front-isometric view of a dual guide sanitisation device 201, similar to the device shown in FIGS. 6-8. As can be seen in FIG. 9, the device comprises a body 202 having two moveable guides 204. Each moveable guide is substantially the same as the moveable guide described above with reference to FIGS. 6-8 unless otherwise stated. The body 202 also comprises a bracket and screw clamp as described above with reference to FIGS. 6-8.

The body 202 can be formed from two sleeves (each sleeve substantially matching the sleeve described above with reference to FIGS. 6-8) integrated together. Alternatively, the body may function as a single sleeve housing two guides 204. The device is provided with a single display 250 for both guides.

The body 202 also comprises two openings 240. These each of these openings expose one or more LEDs or other light sources which are configured to provide a visual indication to the user that sanitisation of a receptacle has been completed. For example, a red LED may be activated when a push button (or other sensor) indicates that a receptacle has been inserted into the device for sanitisation. The red LED may also be coupled to a timer which measures how long the push button (or other sensor) indicates that a receptacle has been inserted into the device for sanitisation. If the timer indicates that the receptacle has been inserted into the device for sanitisation for a sufficient amount of time to sanitise the receptacle, the time can be configured to deactivate the red LED. Alternatively, or in addition, the timer may activate a green LED when the receptacle has been inserted into the device for sanitisation for a sufficient amount of time to sanitise the receptacle. The colours red and green are by way of example, and LEDs may be any suitable colour. Alternative light sources may also be used instead of LEDs.

Of course, other arrangements are possible, and a device comprising more than two moveable guides is also envisaged.

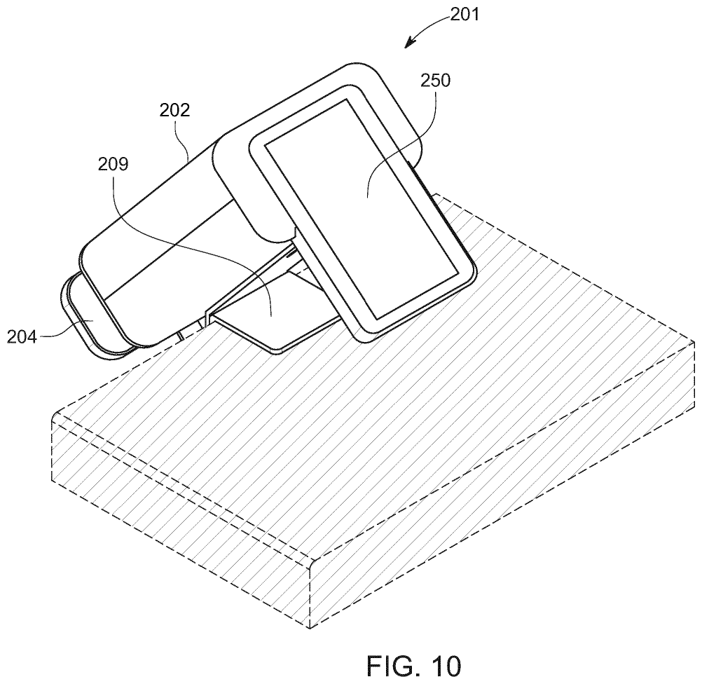

FIG. 10 is a back-isometric view of the dual guide sanitisation device shown in FIG. 9, illustrating the display 250 as it would appear to someone (e.g. a customer) facing the user. This display 250 can show a timer illustrating the remaining time required to sanitise a receptacle upon insertion of the receptacle into the device. The display 250 can also be configured to show a visual warning if the receptacle is removed from the device before it has been correctly sanitised. Alternatively, the device can generate an audio alarm if the receptacle is removed from the device before it has been correctly sanitised.

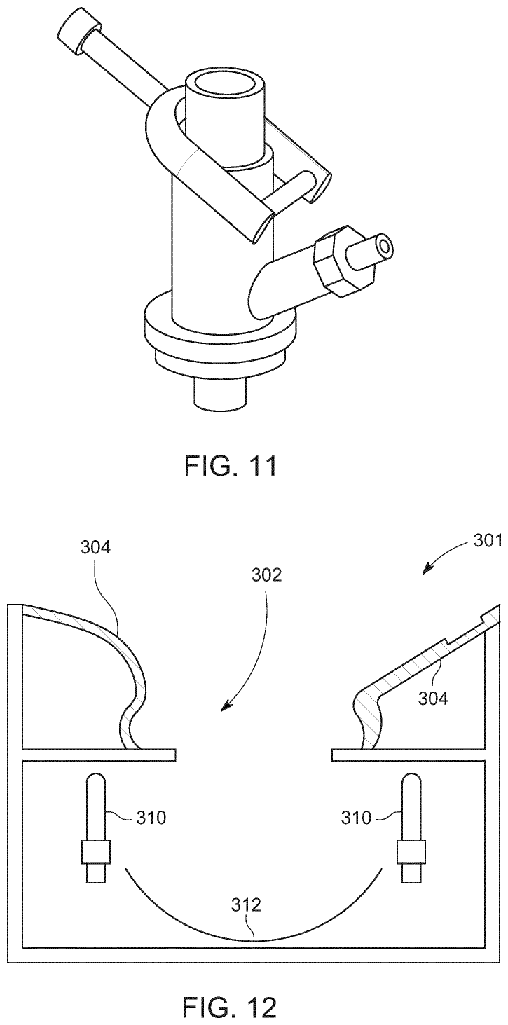

FIG. 11 illustrates a typical keg coupler that connects a keg containing a beverage such as beer to a dispensing line. The keg coupler may be sanitised using a device 301 as illustrated by FIG. 12. The device 301 has an opening 302 comprising a guide 304 shaped to receive the keg coupler. The guide 304 leads to a treatment zone containing a pair of UV bulbs 310 and a reflector 312 arranged to irradiate the surfaces of a keg coupler during use.

Although not shown in the Figures, optically transparent, but UV-opaque, sections can be used to allow a customer to see the UV light in operation. In such an arrangement, a user would only see the visible light emitted by the UV light source(s). Additionally, these sections can optionally be internally coated with a phosphor configured to absorb UV light and emit visible light. Alternatively, or additionally, some form of indicator can be provided (e.g. using visible light emitting LEDs, the display, or an audio alert) to inform an operator when sufficient UV radiation has been applied (e.g. changing colour or turning on/off after 2 seconds).

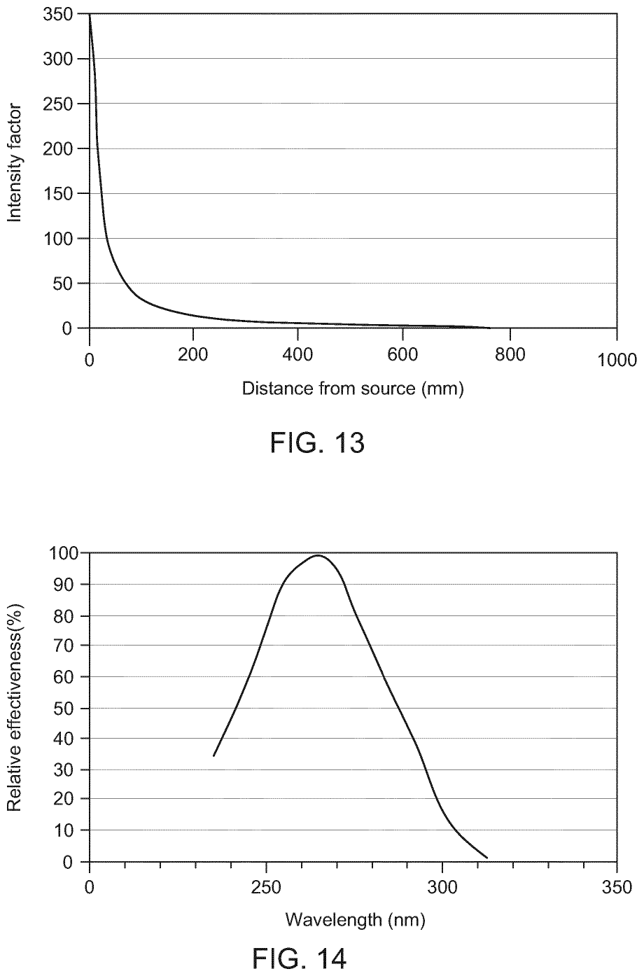

FIG. 13 shows the relationship between the intensity of UV light received by an object from a UV light source and the distance of the object from the UV light source. To achieve a relatively high dose of UV radiation in a relatively short space of time e.g. 1-5 seconds, the surface of a receptacle is preferably positioned within 25 mm of a UV light source.

FIG. 14 shows the spectral distribution of sanitary effectiveness against UV wavelength. Peak sanitary effectiveness occurs between when the UV light has a wavelength of between 263 and 266 nm (IESNA 2000). Therefore, to minimise treatment times, UV light having a wavelength close to this range is preferred. UV light bulb(s) emitting UV light at a wavelength of 254 nm are advantageous because UV light with a wavelength of 253.7 nm is linked to the closest electron energy band available for a mercury lamp.

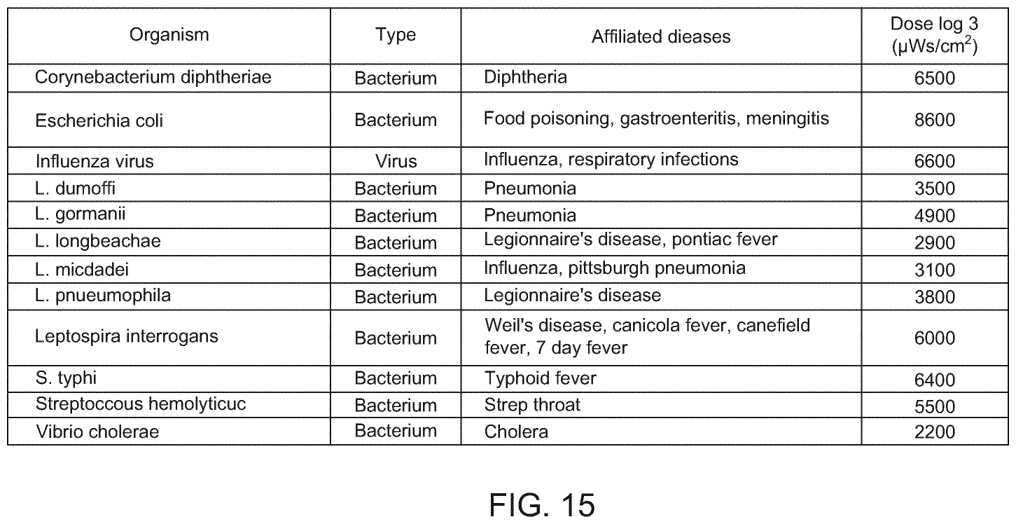

FIG. 15 shows the energy levels of UV light having a wavelength of 254 nm required for the 99.9% (log 3) destruction of various microorganisms. The bacteria Leptospira interrogans, which is the bacteria causing Weil's disease, can be treated with a dose of 6000 μWs/cm² at this wavelength. Weil's disease is known to be passable to humans through drinking from contaminated bottles or cans which have come into contact with vermin excretions. This can occur at any point during the transport and/or storage of a drinks receptacle before it reaches a customer.

It will be readily apparent to those skilled in the art that various modifications can be made to the illustrative examples described above and the generic principles defined herein can be applied to other implementations without departing from the spirit or cope of this disclosure. For example, although the comprises a sleeve, other shapes can be used instead of a sleeve.

As a further example, a sterilising device with a guide shaped to position an opening portion of a drink receptacle (e.g. such as a reusable water bottle) can be integrated with a drink dispenser (such as a water fountain).

Thus, the subject matter for which protection is sought is not limited by any of the foregoing but is instead defined by the appended claims, giving due weight to any equivalents to the features disclosed therein.

1. A sanitisation device having a body, wherein:
the body has an opening configured to receive at least part of a receptacle or tap in use;
the body contains one or more UV light sources; and
the body comprises a guide, wherein the guide is shaped to position an opening portion of the receptacle or tap when the opening portion of the receptacle or tap is inserted into the opening of the body so that the opening portion is correctly located within the body for irradiation with UV light emitted by the one or more UV light sources.

2. The sanitisation device of claim 1, wherein the guide is moveable between a first position and a second position, wherein the guide is configured to activate the one or more UV light sources in the second position.

3. The sanitisation device of any preceding claim, wherein:
the guide is configured to prevent UV light from reaching the exterior of the sanitisation device.

4. The sanitisation device of claim 2 or 3, wherein the guide is provided with a ring of resilient material configured to form a soft seal with the receptacle to prevent UV light reaching a portion of the exterior surface of the receptacle external to the guide in use.

5. The sanitisation device of any one of claims 2-4, wherein the guide comprises a neck shade configured to prevent UV light exiting the device though a receptacle that is at least partially transparent to UV light.

6. The sanitisation device of any one of claims 2-5, wherein the body of the device comprises a collar or one or more notches to prevent the guide being moved beyond the second position.

7. The sanitisation device of any preceding claim wherein the guide is shaped to correctly position at least one of:
a bottle;
a can;
a keg or coupler; or
a tap, wherein a tap is at least one of: a beverage dispensing tap or faucet; a beer tap; or a keg coupler.

8. A device as claimed in any preceding claim, wherein the body is a sleeve and the guide is configured to guide the portion of the exterior surface of the receptacle or the tap so that the portion of the exterior surface is located at a distance of 25 mm or less from the UV light source(s).

9. A device as claimed in claim 8, wherein the distance is at least one of 20 mm, 15 mm, 10 mm, 5 mm, or less than 5 mm from the UV light source(s).

10. A device as claimed in any preceding claim, wherein the guide further comprises shielding means arranged to prevent UV radiation from reaching the opening of the body.

11. A device as claimed in any preceding claim, comprising a first sensing means for detecting the presence of an item correctly located in the body for irradiation with UV light.

12. A device as claimed in any preceding claim, comprising a second sensing means for detecting the presence of an item that is not suitable for treatment with UV light.

13. A point of service for food and/or drink items or a dispensing machine for drink receptacles comprising a sanitisation device according to any preceding claim.

14. A device as claimed in any preceding claim, further comprising reflection means for the one or more UV radiation source(s).

15. A method of sanitising, comprising:
manually inserting a portion of the surface of a receptacle or tap head in a body having an opening; and
activating one or more UV light sources to irradiate the portion of the surface of the receptacle or tap head with UV light, wherein:
the step of manually inserting comprises using a guide to position the portion of the surface of the receptacle or tap to the correct location within the body for irradiation with the UV light emitted by the one or more UV light sources.
